SEQUENCE LISTING (15 sequences)

SEQ ID NO: 1 - DNA, 2891 bp, Homo sapiens
  source 1..2891: mol_type = genomic DNA
tctgacccacagactctccaactctccggcgcttctcgccaactcggtccctctgaacatgaagggctctctcatcctgtcactaaaaagattagctgtcccgaaacacggaaaaagtcgcccctcttctttgcaggattcctcccttgaacttccccaaaccctcttagcgtgacgtgaccccacccctaggtaaccgcagctgcttccttaccagcttcccgcccccggggggcgcctgccggaggccaatgcaaggaccgtccgctaccggctctgccgctatccctgtggggtgaatctaacatggcggataaagacagtaactagtcccctgtttctccgagtgttcgccaagatgattggctctcaccacttgtccctcaaaacgaccacgccattgattggtggagattgcgtcgatggggcggggcagaagcaacctgaacccgaacaacaataacaaacattgaggctgaggggcggaactaggagtgcgcagatgtgggccagagcggatttccccttccccaggcaaattcggcgcccactgcgtccccgcaggccactgaccttagaggactacttgcccgagactcgtggggctggatgggaatcgtagtcttcctaggagttgtaggtatctttttttggcctagtctctgctctcaagataggagaacataacaacactccaatccattactgttgacatgtataagcccgcggaggtctccaatctatccactggatttccgtgagaattgtgcccgctttggtattggatgttcctctccataagactacagtttccaaggaacagtgtggccaaggcctttcgttccgcaatgcatgttggaaatagtagttctttccctccacctcccaacaatccttttatttacctaaactggagacctccattagggcggaaagagtggggtaatgggacctcttcttaagactgctttggacactatcttacgctgatattcaggcctcaggtggcgattctgaccttggtacagcaattactgtgacgtaataagccgcaactggaagcgtagaggcgagagggcgggcgctttacggcgaactcaggtagaattcttccttttccgtctctttctttttatgtcaccaggggaggactgggtggccaacccagagccccgagagatgctaggctctttctgtcccgcccttcctctgactgtgtcttgatttcctattctgagaggctattgctcagcggtttccgtggcaacagtaaagcgtgggaattacagataaattaaaactgtggaacccctttcctcggctgccgccaaggtgttcggtccttccgaggaagctaaggccgcgttggggtgagaccctcacttcatccggtgagtagcaccgcgtccggcagccccagccccacactcgcccgcgctatggcctccgtctcccagcttgcctgcatctactctgccctcattctgcaggactatgaggtgacctttacggaggataagatcaatgcccttattaaagcagccagtgtaaatattgaaactttttggcctggcttgtttgcaaaggtcctggccaacgtcaacattgggagccacatctgcagtgtagagggggggaaaaaaacgtgactgcgcgtcgtgagctcgctgagacgttctggacgggggacaggccgtggggtttctcagataactgggcccctgggctcaggaggcctgcaccctctgctctgggttaaggtagaagagccccgggaaagggacaggggcccaagggatgctccgggggacgggcgggggaaagtgaatttccgaagctaggcagatgggtattcttatgcgaggggcgggggcggaacctgagaggcataaggcgttgtgaaccccccggggaagggggcagtttgtaggtctcgagggaagcactaaggatcaggttgggggcacagtgtgtccgaggaggaatcctcctgataggaactggaatgtgccttgaaggggacaccatgtgtataagaacatcagctggtcgccggggatggtggcttacgcctgtattcctagcactttgggaggccaaggcggatggatcacgaggtcaggagttcgagaccagcctgaccatcgtggtgaaaccccgtctctactaaaaatacaaaaattagccgggcgtggtggcgcgcgccagctactcaggagctgaggcaggagaatcgcttgaacccaggaggcggaggttgcagtgagccgagatcgcgccattgcactccagcctgggtggcagaacgacactccgtctcaaaaacaaacaaagaaataaacaccggctggtatatatgagaagatgggcccttgcggaagaagaagtgccaggaatatgtctgggaaggggaggagacaggattttgtgggagggagaacttaagaactggatccatttgtgctattgagaaagcgcaagagggaagtagaggagcgtcagtagtaacagatgctgccggcagggatgtgcttgagggggatcctgagatgagagtgggtcgctgggaaaggctaggggcagggaggccttgattggtgttggtttggtcgttgttgattttggttttatgcaagaaaaagaaaacagccagaagcattggagaaagctcaccacttacccggtcagtcactcccctgtagctttctctttcttggagaaaggaaaagacccaaagggttggaagcaatatgtgaaaaaatacagaatttatattgtctaattacaaaaagcaacttctagaacctttaaaggattttgtattattctaaaaccttccaaatcttaaatttaccttattttattttatttatttttgagacggagcttcg

SEQ ID NO: 2 - DNA, 464 bp, Homo sapiens
  source 1..464: mol_type = genomic DNA
aaaactgtggaacccctttcctcggctgccgccaaggtgttcggtccttccgaggaagctaaggccgcgttggggtgagaccctcacttcatccggtgagtagcaccgcgtccggcagccccagccccacactcgcccgcgctatggcctccgtctcccagcttgcctgcatctactctgccctcattctgcaggactatgaggtgacctttacggaggataagatcaatgcccttattaaagcagccagtgtaaatattgaaactttttggcctggcttgtttgcaaaggtcctggccaacgtcaacattgggagccacatctgcagtgtagagggggggaaaaaaacgtgactgcgcgtcgtgagctcgctgagacgttctggacgggggacaggccgtggggtttctcagataactgggcccctgggctcaggaggcctgcaccctctgctctgggttaag

SEQ ID NO: 3 - DNA, 433 bp, Homo sapiens
  source 1..433: mol_type = genomic DNA
gggcagtttgtaggtctcgagggaagcactaaggatcaggttgggggcacagtgtgtccgaggaggaatcctcctgataggaactggaatgtgccttgaaggggacaccatgtgtataagaacatcagctggtcgccggggatggtggcttacgcctgtattcctagcactttgggaggccaaggcggatggatcacgaggtcaggagttcgagaccagcctgaccatcgtggtgaaaccccgtctctactaaaaatacaaaaattagccgggcgtggtggcgcgcgccagctactcaggagctgaggcaggagaatcgcttgaacccaggaggcggaggttgcagtgagccgagatcgcgccattgcactccagcctgggtggcagaacgacactccgtctcaaaaacaaacaaagaaataaacaccggctg

SEQ ID NO: 4 - DNA, 15 bp, Homo sapiens
  source 1..15: mol_type = genomic DNA
aacgtgactgcgcgt

SEQ ID NO: 5 - DNA, 15 bp, Synthetic construct
  source 1..15: mol_type = other DNA
acgcgcagtcacgtt

SEQ ID NO: 6 - DNA, 20 bp, Homo sapiens
  source 1..20: mol_type = genomic DNA
aaaacgtgactgcgcgtcgt

SEQ ID NO: 7 - DNA, 20 bp, Synthetic construct
  source 1..20: mol_type = other DNA
acgacgcgcagtcacgtttt

SEQ ID NO: 8 - DNA, 16 bp, Homo sapiens
  source 1..16: mol_type = genomic DNA
catcagctggtcgccg

SEQ ID NO: 9 - DNA, 16 bp, Synthetic construct
  source 1..16: mol_type = other DNA
cggcgaccagctgatg

SEQ ID NO: 10 - DNA, 16 bp, Homo sapiens
  source 1..16: mol_type = genomic DNA
cttgaaggggacacca

SEQ ID NO: 11 - DNA, 16 bp, Synthetic construct
  source 1..16: mol_type = other DNA
tggtgtccccttcaag

SEQ ID NO: 12 - DNA, 16 bp, Homo sapiens
  source 1..16: mol_type = genomic DNA
ggatggtggcttacgc

SEQ ID NO: 13 - DNA, 16 bp, Synthetic construct
  source 1..16: mol_type = other DNA
gcgtaagccaccatcc

SEQ ID NO: 14 - DNA, 451 bp, Homo sapiens
  source 1..451: mol_type = genomic DNA
ccttccctcagctgccgccaagctgctcggtccttccgaggaagctaaggccgcgttggggtaaggccctcacttcatcctgcgactagcaccgcgtccggcagcgcctgccctacactcgcccgtgccatggcctccgtctccaagctcgcctgcatctactcggccctcattctgcacgacgatgaggtgacagtcacggaggataagatcaatgccctcattaaagcagccggtgtaaatgttgaacctttttggcctggcttgtttgcaaaggccctggccaacgtcaacattgggagcctcatctgcaatgtaggggctggtggacctgctccagcagctggtgctgcaccagcaggacgtcctgccccctccactgctgctgctccagctgaggagaagaaagtggaagcaaagaaagaagaatccaaggagtctgatgatgaca

SEQ ID NO: 15 - DNA, 121 bp, Homo sapiens
  source 1..121: mol_type = genomic DNA
aaaactgcgactgcgcggcgtgagctcgctgagacttcctggacgggggacaggctgtggggtttctcagataactgggcccctgcgctcaggaggccttcaccctctgctctgggtaaag

(19) United States
(12) Patent Application Publication
(10) Pub. No.: US 2026/0015616 A1
(43) Pub. Date: Jan. 15, 2026

(54) COMPOSITIONS AND METHODS FOR THE TREATMENT OF CANCER BY TARGETING THE BRCA1 PSEUDOGENE (BRCA1P1)

(71) Applicants: Olufunmilayo I. OLOPADE, Chicago, IL (US); Yoo Jane HAN, Chicago, IL (US); The University of Chicago, Chicago, IL (US)

(72) Inventors: Olufunmilayo I. OLOPADE, Chicago, IL (US); Yoo Jane HAN, Chicago, IL (US)

(21) Appl. No.: 18/880,493

(22) PCT Filed: Jun. 30, 2023
(86) PCT No.: PCT/US2023/069508

§ 371 Date: Dec. 31, 2024

Related U.S. Application Data
(60) Provisional application No. 63/357,641, filed on Jul. 1, 2022.

(51) Int. Cl. C12N 15/113; A61K 31/7105; A61K 31/713; A61K 45/06; A61P 35/00

(52) U.S. Cl. CPC C12N 15/1135; A61K 31/7105; A61K 31/713; A61K 45/06; A61P 35/00; C12N 2310/11; C12N 2310/3231; C12N 2310/3525

(57) ABSTRACT

Provided herein are compositions and method for the treatment of cancer by inhibiting expression of the breast cancer gene 1 pseudogene 1 (BRCA1P1). In particular, provided herein are nucleic acid inhibitors of BRCA1P1 and methods of use thereof for the treatment and prevention of non-breast cancers.

### CROSS-REFERENCE TO RELATED APPLICATIONS

This application claims the benefit of U.S. Provisional Patent Application No. 63/357,641, filed on Jul. 1, 2022, which is incorporated by reference herein.

### FIELD

Provided herein are compositions and method for the treatment of cancer by inhibiting expression of the breast cancer gene 1 pseudogene 1 (BRCA1P1). In particular, provided herein are nucleic acid inhibitors of BRCA1P1 and methods of use thereof for the treatment and prevention of non-breast cancers.

### BACKGROUND

The roles of the BRCA1 tumor suppressor in regulating homologous recombination and DNA damage repair have been extensively studied, but the biological relevance of the BRCA1 ‘pseudogene’ (BRCA1P1) is not well understood, and least partially because such pseudogenes have long been considered non-functional artifacts (i.e., “junk DNA”) of the human genome.

### SUMMARY

Provided herein are compositions and method for the treatment of cancer by inhibiting expression of the breast cancer gene 1 pseudogene 1 (BRCA1P1). In particular, provided herein are nucleic acid inhibitors of BRCA1P1 and methods of use thereof for the treatment and prevention of non-breast cancers.

In some embodiments, provided herein are methods of treating or preventing (e.g., reducing the risk of) a non-breast cancer in a subject comprising administering an inhibitor of BRCA1P1 pseudogene expression or activity to a subject in need thereof. In some embodiments, the subject suffers from a non-breast cancer. In some embodiments, the subject suffers from ovarian cancer, lung cancer, pancreatic cancer, colon cancer, prostate cancer, melanoma, bladder cancer, non-Hodgkin lymphoma, renal cancer, endometrial cancer, pelvis cancer, leukemia, thyroid cancer, or liver cancer. In some embodiments, the subject is at elevated risk of a non-breast cancer based on one or more risk factors. In some embodiments, the risk factor is selected from: a family history of cancer, being in remission form cancer, environmental or behavioral risk factors, or having a mutation or susceptibility factor that places the subject at increased cancer risk. In some embodiments, the inhibitor of BRCA1P1 pseudogene expression or activity comprises a nucleic acid that inhibits expression of the BRCA1P1 pseudogene. In some embodiments, the nucleic acid that inhibits expression of the BRCA1P1 pseudogene is capable of hybridizing with a target sequence within the BRCA1P1 pseudogene. In some embodiments, the nucleic acid that inhibits expression of the BRCA1P1 pseudogene is capable of hybridizing to a portion of SEQ ID NO: 1 under physiological conditions. In some embodiments, the nucleic acid that inhibits expression of the BRCA1P1 pseudogene is capable of hybridizing to a portion of SEQ ID NOS: 2 or 3 under physiological conditions. In some embodiments, the nucleic acid that inhibits expression of the BRCA1P1 pseudogene is capable of hybridizing to all or a portion of one of SEQ ID NOS: 4, 6, 8, 10, and 12, under physiological conditions. In some embodiments, the nucleic acid that inhibits expression of the BRCA1P1 pseudogene is an antisense oligonucleotide (ASO), an siRNA, and shRNA, or an element of a Cas/CRISPR system. In some embodiments, the nucleic acid that inhibits expression of the BRCA1P1 pseudogene is an ASO. In some embodiments, the ASO is capable of hybridizing with a target sequence within the BRCA1P1 pseudogene. In some embodiments, the ASO is capable of hybridizing to a portion of SEQ ID NO: 1 under physiological conditions. In some embodiments, the ASO is capable of hybridizing to a portion of SEQ ID NOS: 2 or 3 under physiological conditions. In some embodiments, the ASO is capable of hybridizing to all or a portion of one of SEQ ID NOS: 4, 6, 8, 10, and 12, under physiological conditions. In some embodiments, the ASO comprises at least 70% (e.g., 70%, 75%, 80%, 85%, 90%, 95%, 100%, or ranges therebetween) sequence identity to one of SEQ ID NOS: 5, 7, 9, 11, and 13. In some embodiments, the ASO comprises one or more chemical modifications. In some embodiments, the ASO comprises a locked nucleic acid (LNA) modification and/or 2′-methoxyethyl (MOE) modification. In some embodiments, the inhibitor is co-administered with a chemotherapeutic, immunotherapeutic, surgery, and/or radiation.

In some embodiments, provided herein are compositions comprising an ASO inhibitor of BRCA1P1. In some embodiments, the ASO is capable of hybridizing with a target sequence within the BRCA1P1 pseudogene. In some embodiments, the ASO is capable of hybridizing to a portion of SEQ ID NO: 1 under physiological conditions. In some embodiments, the ASO is capable of hybridizing to a portion of SEQ ID NOS: 2 or 3 under physiological conditions. In some embodiments, the ASO is capable of hybridizing to all or a portion of one of SEQ ID NOS: 4, 6, 8, 10, and 12, under physiological conditions. In some embodiments, the ASO comprises at least 70% (e.g., 70%, 75%, 80%, 85%, 90%, 95%, 100%, or ranges therebetween) sequence identity to one of SEQ ID NOS: 5, 7, 9, 11, and 13. In some embodiments, the ASO comprises one or more chemical modifications. In some embodiments, the ASO comprises a locked nucleic acid (LNA) modification and/or 2′-methoxyethyl (MOE) modification.

### BRIEF DESCRIPTION OF THE DRAWINGS

FIG. 1. BRCA1P1 Expression in Various Cancer Cells, qRT-PCR was performed in nine cancer cell lines using 18S rRNA expression as an endogenous control. The fold increase was calculated relative to the expression in human mammary epithelial cells (HMEC). Norm and Endo indicate normal breast tissue and endometrial cancer, respectively.

FIG. 2. Increased Apoptosis in BRCA1P1-ASO-Treated Cancer Cells. Apoptosis of BRCA1P1-ASO and control-ASO treated cells was analyzed in six cell lines, using the IncuCyte Live-Cell Imaging System. Apoptosis was quantified using green fluorescent signals from Caspase-3/7 positive apoptotic cells. Data represent the mean and SD of n=4 to 6 biological replicates, and are representative of at least two independent experiments.

FIG. 3. Increased Sensitivity to Doxorubicine in BRCA1P1-ASO-Treated Cancer Cells. Apoptosis of cancer cells treated with doxorubine (DXR) in the presence of absence of BRCA1P1-ASO was analyzed in four cell lines, using the IncuCyte Live-Cell Imaging System, as described in FIG. 2. DXR was applied one day after ASO treatment.

FIG. 4. Increased Sensitivity to TNF-α in BRCA1P1-ASO-Treated Cancer Cells. Apoptosis of cancer cells treated with TNF-α in the presence of absence of BRCA1P1-ASO was analyzed in H522 (lung cancer) cells, using the IncuCyte Live-Cell Imaging System, as described in FIG. 2. TNF-α was applied one day after ASO treatment.

FIG. 5A-B. The Effect of BRCA1P1 (BP1 in short) knock-down in tumor PDOs. (A) PODs were transfected with BP1-ASO using an electroporation system. The transfection efficiency was calculated using the green fluorescence signal from GFP-expressing plasmids. Antiviral gene expression was measured by qRT-PCR. (B) Gene expression data from S030 (ER-positive, lung metastasis) and S021 (TNBC, primary tumor) organoids. The levels of BRCA1P1, MDA-5 (IFIH1) and TNF-α (TNF) transcripts were analyzed in BP1-ASO treated PDOs relative to those in control-ASO (con-ASO) treated groups. All data represent mean and SD of n=3-12 biological replicates (*p<0.05; **p<0.01; ****p<0.0001).

FIG. 6A-C. Regulation of Antiviral Gene Promoter Activities by BRCA1P1-RNAs through the NF-κB subunit, RelA. (A/B). Regulation of antiviral gene expression by BRCA1P1-RNA (BP1-RNA in short); IFN-β stimulates RelA binding to the promoter and increases luciferase activity (A). However, BP1-RNA interferes RelA binding to the promoter and decreases luciferase activity (B). ISRE and NF-κB represent response elements (RE) for IFN-stimulated genes (ISG) and NF-κB, respectively. (C) Luciferase activity assays in HCC-1806 cells showed an increase in the activity with IFN-β, which were diminished with a full length BP1-RNA, not with a fragment of BP1-RNA (exon 1b). All data represent mean and SD of n=3 biological replicates.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a BRCA1P1 inhibitor” is a reference to one or more BRCA1P1 inhibitors and equivalents thercof known to those skilled in the art, and so forth.

As used herein, the term “comprise” and linguistic variations thereof denote the presence of recited feature(s), element(s), method step(s), etc, without the exclusion of the presence of additional feature(s), element(s), method step(s), etc. Conversely, the term “consisting of” and linguistic variations thereof, denotes the presence of recited feature(s), element(s), method step(s), etc, and excludes any unrecited feature(s), element(s), method step(s), etc., except for ordinarily-associated impurities. The phrase “consisting essentially of” denotes the recited feature(s), element(s), method step(s), etc, and any additional feature(s), element(s), method step(s), etc, that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “pharmaceutically acceptable carrier” refers to non-toxic solid, semisolid, or liquid filler, diluent, encapsulating material, formulation auxiliary, excipient, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. A “pharmaceutical composition” typically comprises at least one active agent (e.g., the copolymers described herein) and a pharmaceutically acceptable carrier.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., pharmaceutical compositions of the present invention) to a subject or in vivo, in vitro, or ex vivo cells, tissues, and organs. Exemplary routes of administration to the human body can be through the eyes (e.g., intraocularly, intravitreally, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intrapcritoncally, etc.) and the like.

As used herein, the terms “co-administration” and “co-administer” refer to the administration of at least two agent(s) or therapies to a subject. In some embodiments, the co-administration of two or more agents or therapies is concurrent (e.g., in the same or separate formulations). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the terms “BRCA1P1 pseudogene inhibitor,” “inhibitor of BRCA1P1,” “BRCA1P1 inhibitory agent,” and the like refer to any compositions or components thereof capable of inhibiting the expression or activity of the BRCA1P1 pseudogene or expression products thereof. An “inhibitor of BRCA1P1 pseudogene expression,” “BRCA1P1 pseudogene expression inhibitor,” or the like refers to an agent (e.g., nucleic acid (e.g., antisense oligonucleotide, etc.), etc.) capable if inhibiting the expression of the BRCA1P1 pseudogene.

### DETAILED DESCRIPTION

Provided herein are compositions and method for the treatment of cancer by inhibiting expression of the breast cancer gene 1 pseudogene 1 (BRCA1P1). In particular, provided herein are nucleic acid inhibitors of BRCA1P1 and methods of use thereof for the treatment and prevention of non-breast cancers.

Experiments conducted during development of embodiments herein demonstrated the functional importance of the BRCA1P1 pseudogene in regulating innate immunity and tumor progression in breast cancer. It was found that BRCA1P1 expresses a long-noncoding RNA (lncRNA) in breast cancer cells, which was highly expressed in breast tumors compared to normal breast tissues. Inhibition of BRCA1P1 expression using antisense oligonucleotides (ASO) or CRISPR-Cas9 genome editing tools increased host innate immune responses and restricted virus replication (specifically replication of Sendi virus) in breast cancer cells. Furthermore, depletion of BRCA1P1 induced cancer cell death (apoptosis) through accumulation of reactive oxygen species (ROS) and DNA damage. Accordingly, BRCA1P1-depleted breast cancer cells were more sensitive to genotoxic drugs, with increased apoptosis after doxorubicin and camptothecin treatment. Mechanistically. lncRNA BRCA1P1 is localized in the nucleus of breast cancer cells, binds to the NF-κB subunit RelA, and negatively regulates antiviral gene expression. In a xenograft mouse model of breast cancer, depletion of BRCA1P1 stimulated cytokine expression and local immunity, and suppressed tumor growth. These results indicate an important role for BRCA1P1 in innate immune defense mechanisms and anti-tumor responses in breast cancer cells.

Experiments were conducted during development of embodiments herein to determine (1) whether a role for the BRCA1P1 pseudogene exists in regulating innate immunity and tumor progression in other (non-breast) cancers, and (2) the effect of inhibiting BRCA1P1 expression (e.g., by antisense oligonucleotide) on cancer cell death and sensitivity to chemotherapeutic treatment. The data demonstrate that BRCA1P1 pseudogene functions to regulate innate immunity and tumor progression in diverse cancers (e.g., non-breast cancers (e.g., colon, ovary, endometrial, lung, etc.)) and that inhibiting BRCA1P1 expression stimulates cancer cell death and increases sensitivity to drug treatments.

BRCA1P1, the pseudogene of the BRCA1 tumor suppressor gene, expresses a long-noncoding RNA (lncRNA) through divergent transcription. The lncRNA BRCA1P1 is localized in the nucleus, binds to the NF-κB subunit RelA, and negatively regulates antiviral gene expression. The chromosome 17q21 region containing BRCA1 has a partially duplicated pseudogene, BRCA1P1 (Gene ID: 394269, HUGO ID: 28470), which contains only three of the 24 exons of BRCA1. It also includes an insertion of the acidic ribosomal phosphoprotein P1 pseudogene (RPLP1P4) in exon 1a, displaying unique features of a chimeric pseudogene derived from the two parent genes, BRCA1 and RPLP1. In some embodiments, BRCA1P1 is defined by the NCBI Reference Sequence: NG_003183.3.

Exon 1a and exon 1b sequences specific to BRCA1P1 were selected as antisense targets, avoiding similarity to BRCA1 or RPLP1P4 sequences (scc Table 1 sequence alignment). As BRCA1P1 is a pseudogene of BRCA1, DNA sequences of BRCA1P1 are similar to those of the parent gene BRCA1 (more than 85% homology). However, comparison of DNA sequences between BRCA1 and BRCA1P1 showed three major insertions and several point mutations in BRCA1P1. Exon 1a of BRCA1P1 contains a 343 bp insertion of RPLP1P4, which comprises exons 1-2 and a portion of exon 3 of the RPLP1 gene. ALU elements are also inserted in exon 1b and intron 1b of BRCA1P1. On the basis of this observation, four ASOs were designed (Table 2): one ASO targeting exon 1a (Ex1a-5-LNA or -MOE, depending on chemical modifications) and three ASOs targeting three regions of exon 1b (Ex1b-1,-4, and -17). Out of four ASOs tested, Ex1a-5 ASOs showed the best efficiency in inhibiting BRCA1P1 expression specifically, with no inhibitory effect on BRCA1 or RPLP1 expression. Two chemical modifications were applied to Ex1a-5 ASOs; locked nucleic acid modifications (LNA-Gapmer) with phosphorothioate linkages for 15 nucleotides (6 nucleotides unique to BRCA1P1) and 2′-methoxyethyl (MOE) modifications with phosphorothioate linkages for 20 nucleotides (8 nucleotides unique to BRCA1P1).

TABLE 1
Alignment and Comparison of DNA Sequences of BRCA1-Exon 1a (SEQ ID NO: 15), BRCA1P1-Exon1a (SEQ ID NO:
2) and RPLP1P4 (SEQ ID NO: 14). Exon 1a of BRCA1P1 contains a 343 bp insertion of RPLP1/RPLP1P4 sequences (blue). DNA
sequences mismatched between BRCA1P1 and RPLP1P4 or between BRCA1P1 and BRCA1 are indicated in red. ASO sequences
specific to Exon 1a-5-MOE of BRCA1P1 were boxed (20 nucleotides), whereas Exon 1a-5-LNA sequences are highlighted (15
nucleotides).

| | |
|---|---|
| | ...\|... \| ... \|... \| ...\|...\| ...\|...\| ... \|..\| ...\|...\| ...\|... \|...\|... \|... \|...\|... \|... \| |
| | 10 20 30 40 50 60 70 80 90 100 |
| RPLP1P4 | ---------- -----CTTCC CTCAGCTGCC GCCAAGCTGC TCGGTCCTTC CGAGGAAGCT AAGGCCGCGT TGGGGTAAGG CCCTCACTTC ATCCTGCGAC |
| BRCA1 Ex1a | AAAACTG--- ---------- ---------- ---------- ---------- ---------- ---------- ---------- ---------- ---------- |
| BRCA1P1 Ex1a | AAAACTGTGG AACCCCTTTC CTCGGCTGCC GCCAAGGTGT TCGGTCCTTC CGAGGAAGCT AAGGCCGCGT TGGGGTGAGA CCCTCACTTC ATCCGGTGAG |
| | ...\|... \| ... \|... \| ...\|... \| ...\|... \| ... \|...\| ...\|...\| ...\|... \|...\|... \|... \|... \|... \|...\| |
| | 110 120 130 140 150 160 170 180 190 200 |
| RPLP1P4 | TAGCACCGCG TCCGGCAGCG CCTGCCCTAC ACTCGCCCGT GCCATGGCCT CCGTCTCCAA GCTCGCCTGC ATCTACTCGG CCCTCATTCT GCACGACGAT |
| BRCA1 Ex1a | ---------- ---------- ---------- ---------- ---------- ---------- ---------- ---------- ---------- ---------- |
| BRCA1 Ex1a | TAGCACCGCG TCCGGCAGCC CCAGCCCCAC ACTCGCCCGC GCTATGGCCT CCGTCTCCCA GCTTGCCTGC ATCTACTCTG CCCTCATTCT GCAGGACTAT |
| | ...\|... \| ... \|... \| ...\|... \| ...\|... \| ... \|...\| ...\|...\| ...\|... \|...\|... \|... \|... \|...\|..\| |
| | 210 220 230 240 250 260 270 280 290 300 |
| RPLP1P4 | GAGGTGACAG TCACGGAGGA TAAGATCAAT GCCCTCATTA AAGCAGCCGC TGTAAATGTT GAACCTTTTT GGCCTGGCTT GTTTGCAAAG GCCCTGGCCA |
| BRCA1 Ex1a | ---------- ---------- ---------- ---------- ---------- ---------- ---------- ---------- ---------- ---------- |
| BrCA1 Ex1a | GAGGTGACCT TTACGGAGGA TAAGATCAAT GCCCTTATTA AAGCAGCCAG TGTAAATATT TAAACTTTTT GGCCTGGCTT GTTTGCAAAG GTCCTGGCCA |
| | ...\|... \| ... \|... \| ...\|... \| ...\|... \| ... \|...\| ...\|...\| ...\|... \|...\|... \|... \|...\|...\|...\| |
| RPLP1P4 | 310 320 330 340 350 360 370 380 390 400 |
| BRCA1 Ex1a | ACGTCAACAT TGGGAGCCTC ATCTGCAATG TAGGGGCTGG TGGACCTGCT CCAGCAGCTG GTGCTGCACC AGCAGGACGT CCTGCCCCCT CCACTGCTGC |
| BRCA1P1 Ex1a | 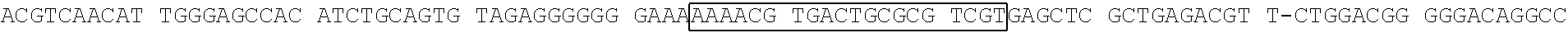 |
| | Ex1a-5-MOE (20-mer); Ex1a-5-LNA (15-mer) |
| | ...\|... \| ... \|... \| ...\|... \| ...\|... \| ... \|...\| ...\|...\| ..\| |
| | 410 420 430 440 450 460 |
| RPLP1P4 | TGCTCCAGCT GAGGAGAAGA AAGTGGAAGC AAAGAAAGAA GAATCCAAGG AGTCTGATGA TGACA |
| BRCA1 Ex1a | CTCCCCTTTC TCACATAACT CCCCCCCTCC CCTCACCACC CCTTCACCCT CTCCTCTCCC TAAAC |
| BRCA1P1 Ex1a | GTGGGGTTTC TCAGATAACT GGGCCCCTGG GCTCAGGAGG CCTGCACCCT CTGCTCTGGG TTAAG |

TABLE 2
DNA sequences of ASOs

| | Target | | | |
|---|---|---|---|---|
| Name | Exon | Target Sequence | ASO Sequence | Modification |
| Ex1a-5- | Exon 1a | AACGTGACTGCGCGT | ACGCGCAGTCACGTT | Locked |
| LNA | | (SEQ ID NO: 4) | (SEQ ID NO: 5) | nucleic acid |
| | | | | (LNA) |
| Ex1a-5- | Exon 1a | AAAACGTGACTGCGCG | ACGACGCGCAGTCAC | 2′- |
| MOE | | TCGT (SEQ ID | GTTTT (SEQ ID NO: 7) | methoxyethyl |
| | | NO: 6) | | (MOE) |
| Ex1b-1 | Exon 1b | CATCAGCTGGTCGCCG | CGGCGACCAGCTGAT | LNA |
| | | (SEQ ID NO: 8) | G (SEQ ID NO: 9) | |
| Ex1b-4 | Exon 1b | CTTGAAGGGGACACCA | TGGTGTCCCCTTCAA | LNA |
| | | (SEQ ID NO: 10) | G (SEQ ID NO: 11) | |
| Ex1b-17 | Exon 1b | GGATGGTGGCTTACGC | GCGTAAGCCACCATC | LNA |
| | | (SEQ ID NO: 12) | C (SEQ ID NO: 13) | |

*underlined are identical sequences between LNA and MOE modified Ex1a-5-ASOs. The MOE modified ASO has additional 5 nucleotides (three and two nucleotides at 5′ and 3′, respectively), to incorporate MOE modifications.

For Ex1a-5-ASOs, exon 1a sequences unique to BRCA1P1 were selected and targeted for inhibition (Table 1). The sequences were selected because there are 6-8 nucleotides unique to BRCA1P1 that are not present in BRCA1 nor RPLP1P4 sequences (see the sequences below), and targeting this site produces a strong inhibition effect on BRCA1P1 expression.

In some embodiments, provided herein are inhibitors of BRCA1P1 expression. In some embodiments, an inhibitor is a small molecule, peptide, antibody, antibody fragment, a genome editing agent, etc. In particular embodiments, a BRCA1P1 inhibitor is a nucleic acid-based inhibitor. In some embodiments, the inhibitor is a small molecule, an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, a transcription activator-like (TAL) effector (TALE) nuclease, etc.

In some embodiments, the BRCA1P1 inhibitor is a small interfering RNA (siRNA), also known as short interfering RNA or silencing RNA. In some embodiments, an siRNA is an 18 to 30 nucleotide, preferably 19 to 25 nucleotide, most preferred 21 to 23 nucleotide or even more preferably 21 nucleotide-long double-stranded RNA molecule, siRNA is involved in the RNA interference (RNAi) pathway where the siRNA interferes with the expression of a specific gene (e.g., the BRCA1P1 pseudogene), siRNAs naturally found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest (e.g., the BRCA1P1 pseudogene). Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, in some embodiments at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA and inhibit the BRCA1P1 pseudogene is envisioned in the present invention. In some embodiments, siRNA duplexes are provided composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair. 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP). In some embodiments, provided herein are siRNA molecules that target and inhibit the expression (e.g., knock down) of the BRCA1P1 pseudogene

A short hairpin RNA (shRNA) is a sequence of RNA that makes a tight hairpin turn that can be used to silence gene expression (e.g., of BRCA1P1) via RNA interference. In some embodiments, shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). The RISC binds to and cleaves RNAs which match the siRNA that is bound to (e.g., comprising the sequence of the BRCA1P1 pseudogene). In some embodiments, si/shRNAs to be used in the present invention are chemically synthesized using appropriately protected ribonucleoside phosphoramidites and a conventional DNA/RNA synthesizer. In some embodiments, provided herein are shRNA molecules that target and inhibit the expression (e.g., knock down) of the BRCA1P1 pseudogene.

Further molecules effecting RNAi (and useful herein for the inhibition of expression of the BRCA1P1 pseudogene) include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of the BRCA1P1 pseudogene after introduction into target cells. In some embodiments, provided herein are miRNA molecules that target and inhibit the expression (e.g., knock down) of the BRCA1P1 pseudogene.

Morpholinos (or morpholino oligonucleotides) are synthetic nucleic acid molecules having a length of about 20 to 30 nucleotides and, typically about 25 nucleotides. Morpholinos bind to complementary sequences of target transcripts (e.g., the BRCA1P1 pseudogene) by standard nucleic acid base-pairing. They have standard nucleic acid bases which are bound to morpholine rings instead of deoxyribose rings and linked through phosphorodiamidate groups instead of phosphates. Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and prevent access by molecules that might otherwise interact with the RNA. In some embodiments, provided herein are morpholino oligonucleotides that target and inhibit the expression (e.g., knock down) of the BRCA1P1 pseudogene.

A ribozyme (ribonucleic acid enzyme, also called RNA enzyme or catalytic RNA) is an RNA molecule that catalyzes a chemical reaction. Many natural ribozymes catalyze cither their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established. Since it was shown that hammerhead structures can be integrated into heterologous RNA sequences and that ribozyme activity can thereby be transferred to these molecules, catalytic antisense sequences can be engineered for almost any target sequence can be created, provided the target sequence contains a potential matching cleavage site. The basic principle of constructing hammerhead ribozymes is as follows: A region of interest of the RNA (e.g., a portion of the BRCA1P1 pseudogene), which contains the GUC (or CUC) triplet, is selected. Two oligonucleotide strands, each usually with 6 to 8 nucleotides, are taken and the catalytic hammerhead sequence is inserted between them. In some embodiments, provided herein are ribozyme inhibitors oligonucleotides of the BRCA1P1 pseudogene.

In some embodiments, BRCA1P1 is inhibited (and/or BRCA1P1 activity is inhibited) by modifying the BRCA1P1 sequence in target cells. In some embodiments, the alteration of the BRCA1P1 pseudogene is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g, tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes. In some aspects, a Cas nuclease and gRNA (including a fusion of crRNA specific for the target sequence (e.g., a sequence within the BRCA1P1 pseudogene) and fixed tracrRNA) are introduced into the cell. In general, target sites at the 5′ end of the gRNA target the Cas nuclease to the target site, e.g., the BRCA1P1 pseudogene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence (e.g., sequence within the BRCA1P1 pseudogene). In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system can induce double stranded breaks (DSBs) at the SRC-3 target site. followed by disruptions or alterations as discussed herein. In other embodiments, Cas9 variants, deemed “nickases,” are used to nick a single strand at the target site (e.g., within the BRCA1P1 pseudogene). Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression (e.g., to inhibit expression of the BRCA1P1 pseudogene). In some embodiments, the CRISPR system is used to alter the BRCA1P1 pseudogene, inhibit expression of the BRCA1P1 pseudogene, and/or to inactivate the expression product of the BRCA1P1 pseudogene.

The term “antisense nucleic acid molecule” or “antisense oligonucleotide” as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901). In some embodiments, provided herein are antisense oligonucleotides capable of inhibiting expression of BRCA1P1 when administered to cell or subject. In some embodiments, the antisense oligonucleotides are antisense DNA-and/or RNA-oligonucleotides. In some embodiments, provided herein are modified antisense oligonucleotides, such as, antisense 2′-O-methyl oligo-ribonucleotides, antisense oligonucleotides containing phosphorothiaote linkages, antisense oligonucleotides containing Locked Nucleic Acid LNA(R) bases, morpholino antisense oligonucleotides, PPAR-gamma agonists, antagomirs. In some embodiments. ASOs comprise Locked Nucleic Acid (LNA) or 2′-methoxyethyl (MOE) modifications (internucleotide linkages are phosphorothioates interspersed with phosphodiesters, and all cytosine residues are 5′-methylcytosines).

Certain embodiments herein are directed to administration of an inhibitor of BRCA1P1 expression to a subject with cancer, in remission from cancer, or at elevated risk of cancer (e.g., a non-breast cancer). In some embodiments, an inhibitor of BRCA1P1 expression is administered as part of therapeutic or prophylactic regimen for the treatment or prevention of acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple mycloma/plasma cell neoplasm, mycosis fungoides, myclodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, the cancer to be treated or prevented is not breast cancer.

Experiments conducted during development of embodiments herein demonstrate that inhibition of the BRCA1P1 pseudogene induced cancer cell death, and in particular, produced a synergistic effect when BRCA1P1 pseudogene expression was inhibited along with a second anti-cancer therapy or therapeutic. As such, in some embodiments, an inhibitor of BRCA1P1 pseudogene expression (or activity) is co-administered with one or more other agents (or therapies) for the treatment/prevention of cancer. In some embodiments, an inhibitor of BRCA1P1 pseudogene expression (or activity) is co-administered with a chemotherapeutic, immunotherapeutic, radiation, surgery, etc.

In some embodiments, BRCA1P1 pseudogene expression (or activity) is inhibited (e.g., by administration of a BRCA1P1 pseudogene inhibitor agent described herein) along with administration of a chemotherapy agent. In some embodiments, the chemotherapeutic is selected from the group consisting of mitotic inhibitors, alkylating agents, anti-metabolites, intercalating antibiotics, growth factor inhibitors, cell cycle inhibitors, enzyme inhibitors, topoisomerase inhibitors, protein-protein interaction inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trictylenephosphoramide, triethylenethiophosphaoramide and trimethylolomelamine; nitrogen mustards such as chlorambucil, chlornaphazine, cholophosphamide, estramustine, ifosfamide, mechlorethamine. mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azascrine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran: spirogermanium; tenuazonic acid; triaziquone; 2,2′,2″-trichlorotricthylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; espcramicins; capccitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; ctoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

Embodiments herein further relate to methods for using a BRCA1P1 inhibitor in combination with radiation therapy for inhibiting abnormal cell growth or treating the hyperproliferative disorder in the mammal. Techniques for administering radiation therapy are known in the art, and these techniques can be used in the combination therapy described herein. The administration of a BRCA1P1 inhibitor in this combination therapy can be determined as described herein. Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

A BRCA1P1 inhibitor may also be used in combination with an amount of one or more substances selected from anti-angiogenesis agents, signal transduction inhibitors. antiproliferative agents, glycolysis inhibitors, or autophagy inhibitors.

Anti-angiogenesis agents, such as MMP-2 (matrix-metalloproteinase 2) inhibitors, MMP-9 (matrix-metalloprotienase 9) inhibitors, and COX-11 (cyclooxygenase 11) inhibitors, can be used in conjunction with a BRCA1P1 inhibitor. Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (e.g., MAP-1, MMP-3, MMP-4, MMP-5, MMP-6, MMP-7, MMP-8, MMP-10, MMP-11, MMP-12, and MMP-13). Some specific examples of MMP inhibitors useful in the invention are AG-3340, RO 32-3555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In some embodiments, medicaments which are administered in conjunction with a BRCA1P1 inhibitor include any suitable drugs usefully delivered by inhalation for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines or pentamidine; antihistamines, e.g., methapyrilene; anti-inflammatoires, e.g., beclomethasone, flunisolide, budesonide, tipredane, triamcinolone acetonide or fluticasone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoctharinc, tulobuterol, orciprenaline or (-)-4-amino-3,5-dichloro-α-[[[6-[2-(2-pyridinyl)ethoxy]hexyl]-amino]methyl]benzenemethanol; diuretics, e.g., amiloride; anticholinergics e.g., ipratropium, atropine or oxitropium; hormones, e.g., cortisone, hydrocortisone or prednisolone; xanthines e.g., aminophylline, choline theophyllinate, lysine theophyllinate or theophylline; and therapeutic proteins and peptides, e.g., insulin or glucagon

Exemplary therapeutic agents useful for a combination therapy with a BRCA1P1 inhibitor include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Other suitable therapeutic agents for coadministration with a BRCA1P1 inhibitor also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated for co-administration with a BRCA1P1 inhibitor include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Other therapeutic agents contemplated for co-administration with a BRCA1P1 inhibitor include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, β-lactam antibiotics, an agent comprising an aminoglycoside, protein synthesis inhibitors, drugs used in the chemotherapy of tuberculosis, mycobacterium avium complex disease, and leprosy, antifungal agents, antiviral agents including nonretroviral agents and antiretroviral agents.

Examples of therapeutic antibodies that can be combined with a BRCA1P1 inhibitor include but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

For treating cancers such as renal carcinoma, one may combine a BRCA1P1 inhibitor with sorafenib and/or avastin. For treating, for example, an endometrial disorder, one may combine a BRCA1P1 inhibitor with doxorubincin, taxotere (taxol), and/or cisplatin (carboplatin). For treating, for example, ovarian cancer, one may combine a BRCA1P1 inhibitor with cisplatin (carboplatin), taxotere, doxorubincin, topotecan, and/or tamoxifen. In some embodiments, one may combine a BRCA1P1 inhibitor with taxotere (taxol), gemcitabinc (capccitabinc), tamoxifen, letrozole, tarceva, lapatinib, PD0325901, avastin, herceptin, OSI-906, and/or OSI-930. For treating, for example, lung cancer, one may combine a compound of the present invention with taxotere (taxol), gemcitabine, cisplatin, pemetrexed, Tarceva, PD0325901, and/or avastin.

Further therapeutic agents that can be combined with a compound herein are found in Goodman and Gilman's “The Pharmacological Basis of Therapeutics” Tenth Edition edited by Hardman, Limbird and Gilman or the Physician's Desk Reference, both of which are incorporated herein by reference in their entirety.

In some embodiments, a BRCA1P1 inhibitor is co-administered with another therapeutic agent effective in treating leukemia and/or other cancers. In some embodiments, a BRCA1P1inhibitor is co-administered with one or more therapeutic agents approved for the treatment of Acute Lymphoblastic Leukemia (ALL), for example: ABITREXATE (Methotrexate), ADRIAMYCIN PFS (Doxorubicin Hydrochloride), ADRIAMYCIN RDF (Doxorubicin Hydrochloride), ARRANON (Nelarabine), Asparaginase Erwinia chrysanthemi, CERUBIDINE (Daunorubicin Hydrochloride), CLAFEN (Cyclophosphamide), CLOFARABINE, CLOFAREX (Clofarabine), CLOLAR (Clofarabine), Cyclophosphamide, Cytarabine, CYTOSAR-U (Cytarabine), CYTOXAN (Cyclophosphamide), Dasatinib, Daunorubicin Hydrochloride, Doxorubicin Hydrochloride, Erwinaze (Asparaginase Erwinia Chrysanthemi), FOLEX (Methotrexate), FOLEX PFS (Methotrexate), GLEEVEC (Imatinib Mesylate), ICLUSIG (Ponatinib Hydrochloride), Imatinib Mesylate, MARQIBO (Vincristine Sulfate Liposome), Methotrexate, METHOTREXATE LPF (Methorexate), MEXATE (Methotrexate), MEXATE-AQ (Methotrexate), Nelarabine, NEOSAR (Cyclophosphamide), ONCASPAR (Pegaspargase), Pegaspargase, Ponatinib Hydrochloride, RUBIDOMYCIN (Daunorubicin Hydrochloride), SPRYCEL (Dasatinib), TARABINE PFS (Cytarabine), VINCASAR PFS (Vincristine Sulfate), Vincristine Sulfate, etc.

In some embodiments, a BRCA1P1 inhibitor is co-administered with one or more therapeutic agents approved for the treatment of Acute Myeloid Leukemia (AML), for example: ADRIAMYCIN PFS (Doxorubicin Hydrochloride), ADRIAMYCIN RDF (Doxorubicin Hydrochloride), Arsenic Trioxide, CERUBIDINE (Daunorubicin Hydrochloride), CLAFEN (Cyclophosphamide), Cyclophosphamide, Cytarabine, CYTOSAR-U (Cytarabine), CYTOXAN (Cyclophosphamide), Daunorubicin Hydrochloride, Doxorubicin Hydrochloride, NEOSAR (Cyclophosphamide), RUBIDOMYCIN (Daunorubicin Hydrochloride), RYDAPT (Midostaurin), TARABINE PFS (Cytarabine), TRISENOX (Arsenic Trioxide), VINCASAR PFS (Vincristine Sulfate), Vincristine Sulfate, etc.

In some embodiments, a BRCA1P1 inhibitor is co-administered with one or more therapeutic agents approved for the treatment of Chronic Lymphocytic Leukemia (CLL), for example: Alemtuzumab, AMBOCHLORIN (Chlorambucil), AMBOCLORIN (Chlorambucil), ARZERRA (Ofatumumab), Bendamustine Hydrochloride, CAMPATH (Alemtuzumab), CHLORAMBUCILCLAFEN (Cyclophosphamide), Cyclophosphamide, CYTOXAN (Cyclophosphamide), FLUDARA (Fludarabine Phosphate), Fludarabine Phosphate, LEUKERAN (Chlorambucil), LINFOLIZIN (Chlorambucil), NEOSAR (Cyclophosphamide), Ofatumumab, TREANDA (Bendamustine Hydrochloride), etc.

In some embodiments, a BRCA1P1 inhibitor is co-administered with one or more therapeutic agents approved for the treatment of Chronic Myelogenous Leukemia (CML), for example: BOSULIF (Bosutinib), Bosutinib, CLAFEN (Cyclophosphamide), Cyclophosphamide, Cytarabine, CYTOSAR-U (Cytarabine), CYTOXAN (Cyclophosphamide), Dasatinib, GLEEVEC (Imatinib Mesylate), ICLUSIG (Ponatinib Hydrochloride), Imatinib Mesylate, NEOSAR (Cyclophosphamide), Nilotinib, Omacetaxine Mepesuccinate, Ponatinib Hydrochloride, SPRYCEL (Dasatinib), SYNRIBO (Omacetaxine Mepesuccinate), TARABINE PFS (Cytarabine), TASIGNA (Nilotinib), etc.

In some embodiments, a BRCA1P1 inhibitor is co-administered with one or more therapeutic agents approved for the treatment of Meningeal Leukemia, for example: CYTARABINE, CYTOSAR-U (Cytarabine), TARABINE PFS (Cytarabine), etc.

In some embodiments, a BRCA1P1 inhibitor is co-administered with one or more alkylating agents (e.g., for the treatment of cancer) selected from, for example, nitrogen mustard N-oxide, cyclophosphamide, ifosfamide, thiotepa, ranimustine, nimustine, temozolomide, altretamine, apaziquone, brostallicin, bendamustine, carmustine, estramustine, fotemustine, glufosfamide, mafosfamide, bendamustin, mitolactol, cisplatin, carboplatin, eptaplatin, lobaplatin, nedaplatin, oxaliplatin, and satraplatin.

In some embodiments, a BRCA1P1 inhibitor is co-administered with one or more anti-metabolites (e.g., for the treatment of cancer) selected from, for example, methotrexate, 6-mercaptopurineriboside, mercaptopurine, 5-fluorouracil, tegafur, doxifluridine, carmofur, cytarabinc, cytarabinc ocfosfatc, cnocitabine, gemcitabinc, fludarabin, 5-azacitidine, capecitabine, cladribine, clofarabine, decitabine, eflornithine, ethynylcytidine, cytosine arabinoside, hydroxyurea, melphalan, nelarabine, nolatrexed, ocfosf[iota]te, disodium premetrexed, pentostatin, pelitrexol, raltitrexed, triapine, trimetrexate, vidarabine, vincristine, and vinorelbine;

In some embodiments, a BRCA1P1 inhibitor is co-administered with one or more hormonal therapy agents (e.g., for the treatment of cancer) selected from, for example, exemestane. Lupron, anastrozole, doxercalciferol, fadrozole, formestane, abiraterone acetate, finasteride, epristeride, tamoxifen citrate, fulvestrant, Trelstar, toremifene, raloxifene, lasofoxifene, letrozole, sagopilone, ixabepilone, epothilone B, vinblastine, vinflunine, docetaxel, and paclitaxel;

In some embodiments, a BRCA1P1 inhibitor is co-administered with one or more cytotoxic topoisomerase inhibiting agents (e.g., for the treatment of cancer) selected from, for example, aclarubicin, doxorubicin, amonafide, belotecan, camptothecin, 10-hydroxycamptothecin, 9-aminocamptothecin, diflomotecan, irinotecan, topotecan, edotecarin, epimbicin, etoposide, exatecan, gimatecan, lurtotecan, mitoxantrone, pirambicin, pixantrone, rubitecan, sobuzoxane, tafluposide, etc.

In some embodiments, a BRCA1P1 inhibitor is co-administered with one or more anti-angiogenic compounds (e.g., for the treatment of cancer) selected from, for example, acitretin, aflibercept, angiostatin, aplidine, asentar, axitinib, recentin, bevacizumab, brivanib alaninat, cilengtide, combretastatin, DAST, endostatin, fenretinide, halofuginone, pazopanib, ranibizumab, rebimastat, removab, revlimid, sorafenib, vatalanib, squalamine, sunitinib, telatinib, thalidomide, ukrain, and vitaxin.

In some embodiments, a BRCA1P1 inhibitor is co-administered with one or more antibodies (e.g., for the treatment of cancer) selected from, for example, trastuzumab, cetuximab, bevacizumab, rituximab, ticilimumab, ipilimumab, lumiliximab, catumaxomab, atacicept, oregovomab, and alemtuzumab.

In some embodiments a BRCA1P1 inhibitor is co-administered with one or more VEGF inhibitors (e.g., for the treatment of cancer) selected from, for example, sorafenib, DAST, bevacizumab, sunitinib, recentin, axitinib, aflibercept, telatinib, brivanib alaninate, vatalanib, pazopanib, and ranibizumab.

In some embodiments, a BRCA1P1 inhibitor is co-administered with one or more EGFR inhibitors (e.g., for the treatment of cancer) selected from, for example, cetuximab, panitumumab, vectibix, gefitinib, erlotinib, and Zactima.

In some embodiments, a BRCA1P1 inhibitor is co-administered with one or more HER2 inhibitors (e.g., for the treatment of cancer) selected from, for example, lapatinib, tratuzumab, and pertuzumab; CDK inhibitor is selected from roscovitine and flavopiridol;

In some embodiments, a BRCA1P1 inhibitor is co-administered with one or more proteasome inhibitors (e.g., for the treatment of cancer) selected from, for example, bortezomib and carfilzomib.

In some embodiments, a BRCA1P1 inhibitor is co-administered with one or more serine/threonine kinase inhibitors (e.g., for the treatment of cancer), for example, MEK inhibitors and Raf inhibitors such as sorafenib.

In some embodiments, a BRCA1P1 inhibitor is co-administered with one or more tyrosine kinase inhibitors (e.g., for the treatment of cancer) selected from, for example, dasatinib, nilotibib, DAST, bosutinib, sorafenib, bevacizumab, sunitinib, AZD2171, axitinib, aflibercept, telatinib, imatinib mesylate, brivanib alaninate, pazopanib, ranibizumab, vatalanib, cetuximab, panitumumab, vectibix, gefitinib, erlotinib, lapatinib, tratuzumab, pertuzumab and midostaurin

In some embodiments, a BRCA1P1 inhibitor is co-administered with one or more androgen receptor antagonists (e.g., for the treatment of cancer) selected from, for example, nandrolone decanoate, fluoxymesterone, Android, Prostaid, andromustine, bicalutamide, flutamide, apocyproterone, apoflutamide, chlormadinone acetate, Androcur, Tabi, cyproterone acetate, and nilutamide.

In some embodiments, a BRCA1P1 inhibitor described herein is co-administered with one or more aromatase inhibitors (e.g., for the treatment of cancer) selected from, for example, anastrozole, letrozole, testolactone, exemestane, aminoglutethimide, and formestane.

In some embodiments, a BRCA1P1 inhibitor is co-administered with one or more other anti-cancer agents including, e.g., alitretinoin, ampligen, atrasentan bexarotene, borte-zomib, bosentan, calcitriol, exisulind, fotemustine, ibandronic acid, miltefosine, mitoxantrone, 1-asparaginase, procarbazine, dacarbazine, hydroxycarbamide, pegaspargase, pentostatin, tazaroten, velcade, gallium nitrate, canfosfamide, darinaparsin, and tretinoin. In a preferred embodiment, the compounds of the present disclosure may be used in combination with chemotherapy (e.g., cytotoxic agents), anti-hormones and/or targeted therapies such as other kinase inhibitors, mTOR inhibitors and angiogenesis inhibitors.

In some embodiments, a BRCA1P1 inhibitor is co-administered with one or more immunotherapeutics. In some embodiments, a BRCA1P1 inhibitor is co-administered with a T-cell-based immunotherapeutic, some embodiments, a BRCA1P1 inhibitor is co-administered with one or more immunotherapeutics selected from a therapy comprising the administration of immune checkpoint inhibitor, CAR-T cell therapy, monoclonal antibody therapy, and bispecific T-cell engager therapy. In some embodiments, a BRCA1P1 inhibitor is co-administered with an immune checkpoint inhibitor that binds to and inhibits the activity of an immune checkpoint protein is selected from the group consisting of CTLA4, PD-1, PD-L1, PD-L2, A2AR, B7-H3, B7-H4, BTLA, KIR, LAG3, TIM-3 or VISTA. In some embodiments, the a BRCA1P1 inhibitor is co-administered with an immune checkpoint inhibitor selected from the group consisting of nivolumab, pembrolizumab, pidilizumab, AMP-224, AMP-514, STI-A1110, TSR-042, RG-7446, BMS-936559, BMS-936558, MK-3475, MPDL3280A, MEDI-4736, MSB-0020718C, AUR-012 and STI-A1010.

a BRCA1P1 inhibitor may be used in combination with the agents disclosed herein or other suitable agents, depending on the condition being treated. When used in a combination therapy, a BRCA1P1 inhibitor is administered simultaneously or separately with the second agent. This administration in combination can include simultaneous administration of the two agents in the same dosage form, simultaneous administration in separate dosage forms, and separate administration. That is, a BRCA1P1 inhibitor and any of the agents described above can be formulated together in the same dosage form and administered simultaneously. Alternatively, a BRCA1P1 inhibitor and any of the agents described above can be simultaneously administered, wherein both the agents are present in separate formulations. In another alternative, a BRCA1P1 inhibitor can be administered just followed by and any of the agents described above, or vice versa. In some embodiments of the separate administration protocol, a BRCA1P1 inhibitor and any of the agents described above are administered a few minutes apart, or a few hours apart, or a few days apart.

In embodiments in which the a BRCA1P1 inhibitor used for the treatment or prevention of non-cancer discases and/or conditions, a BRCA1P1 inhibitor may be co-administered with therapeutics and/or therapies known in the field to be appropriate for the treatment of such diseases and/or conditions.

In some embodiments, provided herein arc compositions (e.g., pharmaceutical compositions, etc.) comprising a BRCA1P1 inhibitor. In some embodiments, provided herein are compositions (e.g., pharmaceutical compositions, etc.) comprising a BRCA1P1 inhibitor and one or more additional agents (e.g., the agents described above) for the treatment of cancer.

### EXPERIMENTAL

### Example 1

### Determining The Effects of BRCA1P1-ASO on Inducing Cancer Cell Death and Increasing Sensitivity to Chemotherapy Drug

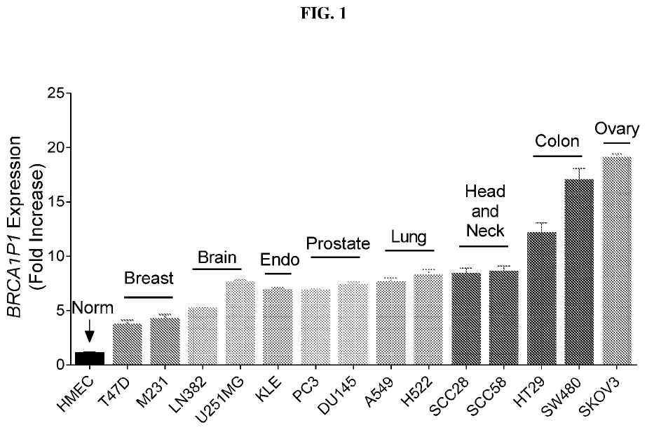

Antisense oligonucleotides (ASO) were developed that specifically inhibit BRCA1P1 transcripts. To determine the effect of BRCA1P1-ASO on diverse cancer cells, BRCA1P1 expression was evaluated in fourteen cancer cell lines representing eight types of cancer (breast, brain, endometrial, prostate, lung, head and neck, colon, and ovarian cancers). While BRCA1P1 expression was 4-fold higher in breast cancer cells (MDA-MB-231) compared to normal breast epithelial cells (HMEC), the expression was much higher in other cancer cell lines: prostate (an 6 to 7-fold increase), lung (an 7 to 9-fold increase), colon (a 11 to 18-fold increase), and ovarian cancer (a 19-fold increase) (FIG. 1). The data indicate that BRCA1P1 is highly expressed in a variety of cancer cells, beyond breast cancer and can be targeted in many types of human cancers.

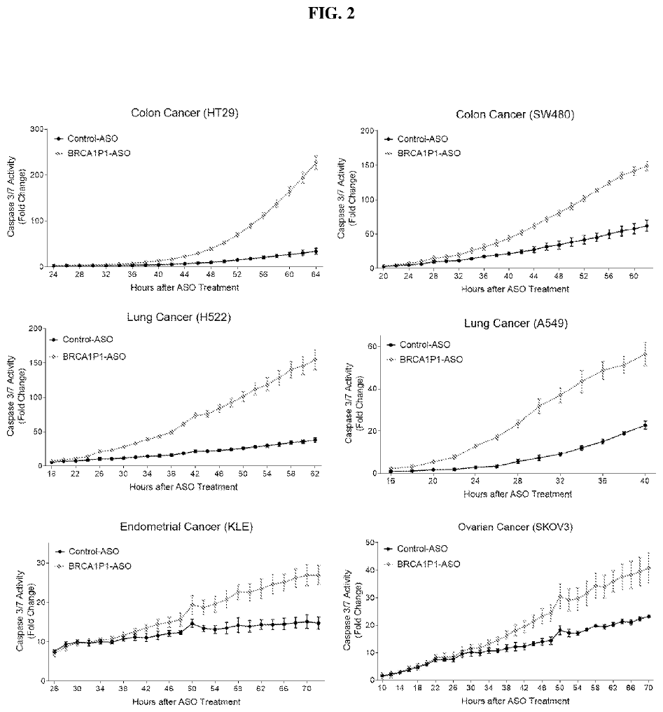

The six cancer cell lines were treated with BRCA1P1-specific ASO or control-ASO. An increased number of caspase 3/7-positive apoptotic cells was observed in BRCA1P1-ASO-treated cells compared to the control-ASO-treated cells in all six cell lines (FIG. 2). The data indicate that BRCA1P1-ASO is effective in killing all types of cancer cells.

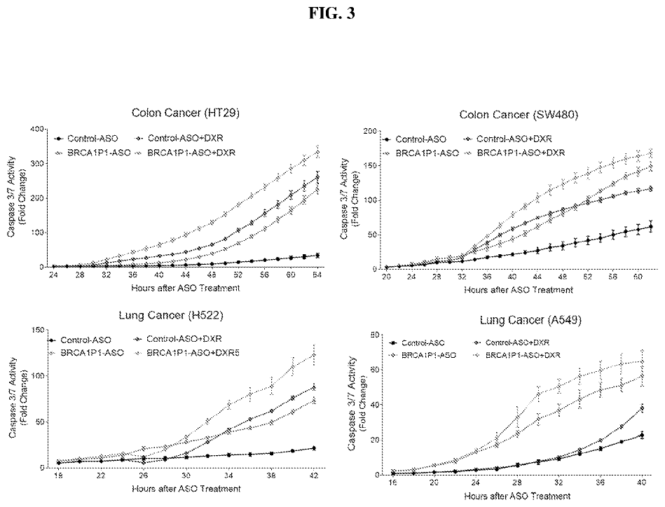

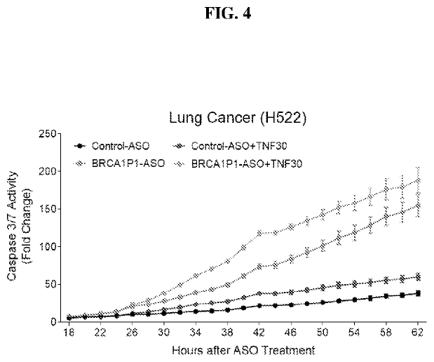

Experiments were conducted during development of embodiments herein to determine whether BRCA1P1-ASO increased sensitivity to chemotherapy drugs such as doxorubicin (Adriamycin). A combined treatment of doxorubicin (DXR) with BRCA1P1-ASO further increased apoptosis of colon and lung cancer cells (FIG. 3). Together, these data indicate that BRCA1P1 depletion increases apoptosis and sensitizes cancer cells to genotoxic drug treatments. BRCA1P1-ASO also increased sensitivity to TNF-α in lung cancer cells, which resulted in increased apoptosis (FIG. 4). Collectively, the data indicate that BRCA1P1-ASO stimulates cancer cell death and increases sensitivity to drug treatments, increasing effectiveness and efficacy of chemotherapy and immunotherapy in various cancers.

### Example 2

Targeting Antiviral and Antitumor immunity in Patient-Derived Breast Tumor Organoids (PDOs) using Antisense Oligonucleotides (ASO) Specific to the BRCA1 Pseudogene (BRCA1P1)

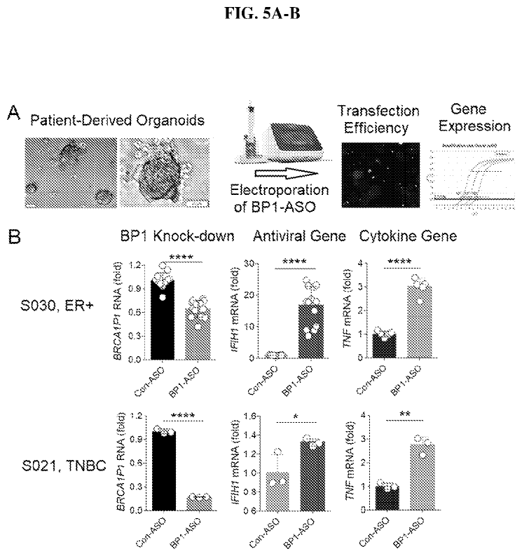

Patient-derived organoids (PDOs) are ex vivo preclinical models that overcome limitations of cell lines or xenograft models, and demonstrate incredible value allowing for modeling and evaluation of treatment response with precision and high-throughput. Experiments were conducted during development of embodiments herein to establish various types (primary or metastatic tumors with diverse subtypes) of breast tumor PDOs and evaluate the therapeutic value of targeting BRCA1P1 in these PDOs (FIG. 5). Antisense oligonucleotides (ASO) specifically inhibiting BRCA1P1 expression were delivered to two PDOs (S030 and S021). S030 and S021 are PDOs established from estrogen receptor (ER)-positive lung metastasis tumors and triple negative breast cancer (TNBC) primary tumors, respectively. Electroporation of BRCA1P1-ASO (BP1-ASO in short) significantly inhibited BRCA1P1-RNA expression in various PDOs with 40-80% knock-down efficiencies (left panel, FIG. 5B). Inhibition of BRCA1P1 with ASO stimulated antiviral (IFIH1) and cytokine (TNF) gene expression, showing 3.0±0.1-fold and 2.7±0.2-fold increases in TNF mRNA expression in the BP1-ASO treated S030 and S021 organoids, respectively, compared to their control-ASO treated organoids (right panel, FIG. 5B).

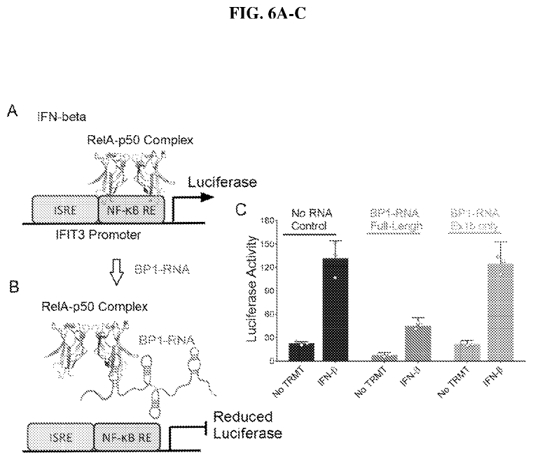

Experiments were conducted during development of embodiments herein to determine the mechanism of how BRCA1P1 transcripts regulate antiviral gene expression. BRCA1P1-RNA (BP1-RNA in short) is localized in the nucleus and binds to the NF-κB subunit RelA, which inhibits the activity of RelA at its target promoters and thereby negatively regulates transcription of antiviral genes. To further dissect the mechanism, the promoter of IFIT3, an IFN-stimulated antiviral gene, was cloned upstream of a luciferase reporter gene, and breast cancer cells were transfected with the constructs (FIG. 6). Experiments demonstrate that treatment of IFN-β stimulates RelA binding to the promoter and activates transcription of luciferase (FIG. 6A). In accordance, we observed a 5.8-fold increase in luciferase activity in IFN-β treated cells compared to no treatment group (left panel, FIG. 6C). However, co-transfection of a full length in vitro transcribed BP1-RNA significantly decreased IFN-β-stimulated luciferase activity from a 5.8-fold to a 2-fold increase (left and middle panels, FIG. 6C), in line with the finding that BP1-RNA interferes with RelA binding to the promoter and thereby reduces luciferase expression (FIG. 6B). In contrast, a fragment (exon 1b only) of BP1-RNA showed no significant changes in IFN-β-stimulated luciferase activity compared to no RNA treated control (a 5.8-fold vs 5.5-fold increase) (left and right panels, FIG. 6C). These results indicate an important role for BRCA1P1 in innate immune defense mechanisms and antitumor responses through the regulation of NF-kB's access to antiviral gene promoters.

The experiments conducted during development of embodiments herein demonstrate the regulation of antiviral/cytokine gene expression by BRCA1P1 in preclinical tumor models. These experiments indicate that BRCA1P1 transcript serves as an immune-dampening RNA and that targeting BRCA1P1 with antisense, small molecule, or other inhibitors boosts antiviral defense mechanisms and antitumor immune responses in tumors.

SEQUENCES

SEQ ID NO: 1-BRCA1P1 [2,891 nucleotides including the promoter and
transcript (exon1a, intron 1a, exon 1b, and a portion of intron 1b)]
TCTGACCCACAGACTCTCCAACTCTCCGGCGCTTCTCGCCAACTCGGTCC
CTCTGAACATGAAGGGCTCTCTCATCCTGTCACTAAAAAGATTAGCTGTC
CCGAAACACGGAAAAAGTCGCCCCTCTTCTTTGCAGGATTCCTCCCTTGA
ACTTCCCCAAACCCTCTTAGCGTGACGTGACCCCACCCCTAGGTAACCGC
AGCTGCTTCCTTACCAGCTTCCCGCCCCCGGGGGGCGCCTGCCGGAGGCC
AATGCAAGGACCGTCCGCTACCGGCTCTGCCGCTATCCCTGTGGGGTGAA
TCTAACATGGCGGATAAAGACAGTAACTAGTCCCCTGTTTCTCCGAGTGT
TCGCCAAGATGATTGGCTCTCACCACTTGTCCCTCAAAACGACCACGCCA
TTGATTGGTGGAGATTGCGTCGATGGGGCGGGGCAGAAGCAACCTGAACC
CGAACAACAATAACAAACATTGAGGCTGAGGGGCGGAACTAGGAGTGCGC
AGATGTGGGCCAGAGCGGATTTCCCCTTCCCCAGGCAAATTCGGCGCCCA
CTGCGTCCCCGCAGGCCACTGACCTTAGAGGACTACTTGCCCGAGACTCG
TGGGGCTGGATGGGAATCGTAGTCTTCCTAGGAGTTGTAGGTATCTTTTT
TTGGCCTAGTCTCTGCTCTCAAGATAGGAGAACATAACAACACTCCAATC
CATTACTGTTGACATGTATAAGCCCGCGGAGGTCTCCAATCTATCCACTG
GATTTCCGTGAGAATTGTGCCCGCTTTGGTATTGGATGTTCCTCTCCATA
AGACTACAGTTTCCAAGGAACAGTGTGGCCAAGGCCTTTCGTTCCGCAAT
GCATGTTGGAAATAGTAGTTCTTTCCCTCCACCTCCCAACAATCCTTTTA
TTTACCTAAACTGGAGACCTCCATTAGGGCGGAAAGAGTGGGGTAATGGG
ACCTCTTCTTAAGACTGCTTTGGACACTATCTTACGCTGATATTCAGGCC
TCAGGTGGCGATTCTGACCTTGGTACAGCAATTACTGTGACGTAATAAGC
CGCAACTGGAAGCGTAGAGGCGAGAGGGCGGGCGCTTTACGGCGAACTCA
GGTAGAATTCTTCCTTTTCCGTCTCTTTCTTTTTATGTCACCAGGGGAGG
ACTGGGTGGCCAACCCAGAGCCCCGAGAGATGCTAGGCTCTTTCTGTCCC
GCCCTTCCTCTGACTGTGTCTTGATTTCCTATTCTGAGAGGCTATTGCTC
AGCGGTTTCCGTGGCAACAGTAAAGCGTGGGAATTACAGATAAATTAAAA
CTGTGGAACCCCTTTCCTCGGCTGCCGCCAAGGTGTTCGGTCCTTCCGAG
GAAGCTAAGGCCGCGTTGGGGTGAGACCCTCACTTCATCCGGTGAGTAGC
ACCGCGTCCGGCAGCCCCAGCCCCACACTCGCCCGCGCTATGGCCTCCGT
CTCCCAGCTTGCCTGCATCTACTCTGCCCTCATTCTGCAGGACTATGAGG
TGACCTTTACGGAGGATAAGATCAATGCCCTTATTAAAGCAGCCAGTGTA
AATATTGAAACTTTTTGGCCTGGCTTGTTTGCAAAGGTCCTGGCCAACGT
CAACATTGGGAGCCACATCTGCAGTGTAGAGGGGGGGAAAAAAACGTGAC
TGCGCGTCGTGAGCTCGCTGAGACGTTCTGGACGGGGGACAGGCCGTGGG
GTTTCTCAGATAACTGGGCCCCTGGGCTCAGGAGGCCTGCACCCTCTGCT
CTGGGTTAAGGTAGAAGAGCCCCGGGAAAGGGACAGGGGCCCAAGGGATG
CTCCGGGGGACGGGCGGGGGAAAGTGAATTTCCGAAGCTAGGCAGATGGG
TATTCTTATGCGAGGGGCGGGGGCGGAACCTGAGAGGCATAAGGCGTTGT
GAACCCCCCGGGGAAGGGGGCAGTTTGTAGGTCTCGAGGGAAGCACTAAG
GATCAGGTTGGGGGCACAGTGTGTCCGAGGAGGAATCCTCCTGATAGGAA
CTGGAATGTGCCTTGAAGGGGACACCATGTGTATAAGAACATCAGCTGGT
CGCCGGGGATGGTGGCTTACGCCTGTATTCCTAGCACTTTGGGAGGCCAA
GGCGGATGGATCACGAGGTCAGGAGTTCGAGACCAGCCTGACCATCGTGG
TGAAACCCCGTCTCTACTAAAAATACAAAAATTAGCCGGGCGTGGTGGCG
CGCGCCAGCTACTCAGGAGCTGAGGCAGGAGAATCGCTTGAACCCAGGAG
GCGGAGGTTGCAGTGAGCCGAGATCGCGCCATTGCACTCCAGCCTGGGTG
GCAGAACGACACTCCGTCTCAAAAACAAACAAAGAAATAAACACCGGCTG
GTATATATGAGAAGATGGGCCCTTGCGGAAGAAGAAGTGCCAGGAATATG
TCTGGGAAGGGGAGGAGACAGGATTTTGTGGGAGGGAGAACTTAAGAACT
GGATCCATTTGTGCTATTGAGAAAGCGCAAGAGGGAAGTAGAGGAGCGTC
AGTAGTAACAGATGCTGCCGGCAGGGATGTGCTTGAGGGGGATCCTGAGA
TGAGAGTGGGTCGCTGGGAAAGGCTAGGGGCAGGGAGGCCTTGATTGGTG
TTGGTTTGGTCGTTGTTGATTTTGGTTTTATGCAAGAAAAAGAAAACAGC
CAGAAGCATTGGAGAAAGCTCACCACTTACCCGGTCAGTCACTCCCCTGT
AGCTTTCTCTTTCTTGGAGAAAGGAAAAGACCCAAAGGGTTGGAAGCAAT
ATGTGAAAAAATACAGAATTTATATTGTCTAATTACAAAAAGCAACTTCT
AGAACCTTTAAAGGATTTTGTATTATTCTAAAACCTTCCAAATCTTAAAT
TTACCTTATTTTATTTTATTTATTTTTGAGACGGAGCTTCG
SEQ ID NO: 2-BRCA1P1-Exon1a (464 nucleotides)
AAAACTGTGGAACCCCTTTCCTCGGCTGCCGCCAAGGTGTTCGGTCCTTC
CGAGGAAGCTAAGGCCGCGTTGGGGTGAGACCCTCACTTCATCCGGTGAG
TAGCACCGCGTCCGGCAGCCCCAGCCCCACACTCGCCCGCGCTATGGCCT
CCGTCTCCCAGCTTGCCTGCATCTACTCTGCCCTCATTCTGCAGGACTAT
GAGGTGACCTTTACGGAGGATAAGATCAATGCCCTTATTAAAGCAGCCAG
TGTAAATATTGAAACTTTTTGGCCTGGCTTGTTTGCAAAGGTCCTGGCCA
ACGTCAACATTGGGAGCCACATCTGCAGTGTAGAGGGGGGGAAAAAAACG
TGACTGCGCGTCGTGAGCTCGCTGAGACGTTCTGGACGGGGGACAGGCCG
TGGGGTTTCTCAGATAACTGGGCCCCTGGGCTCAGGAGGCCTGCACCCTC
TGCTCTGGGTTAAG
SEQ ID NO: 3-BRCA1P1-Exon1b (433 nucleotides)
GGGCAGTTTGTAGGTCTCGAGGGAAGCACTAAGGATCAGGTTGGGGGCAC
AGTGTGTCCGAGGAGGAATCCTCCTGATAGGAACTGGAATGTGCCTTGAA
GGGGACACCATGTGTATAAGAACATCAGCTGGTCGCCGGGGATGGTGGCT
TACGCCTGTATTCCTAGCACTTTGGGAGGCCAAGGCGGATGGATCACGAG
GTCAGGAGTTCGAGACCAGCCTGACCATCGTGGTGAAACCCCGTCTCTAC
TAAAAATACAAAAATTAGCCGGGCGTGGTGGCGCGCGCCAGCTACTCAGG
AGCTGAGGCAGGAGAATCGCTTGAACCCAGGAGGCGGAGGTTGCAGTGAG
CCGAGATCGCGCCATTGCACTCCAGCCTGGGTGGCAGAACGACACTCCGT
CTCAAAAACAAACAAAGAAATAAACACCGGCTG
SEQ ID NO: 4-Exon 1a target 1
AACGTGACTGCGCGT
SEQ ID NO: 5-ASO Ex1a-5-LNA
ACGCGCAGTCACGTT
SEQ ID NO: 6-Exon 1a target 2
AAAACGTGACTGCGCGTCGT
SEQ ID NO: 7-ASO Ex1a-5-MOE
ACGACGCGCAGTCACGTTTT
SEQ ID NO: 8-Exon 1b target 1
CATCAGCTGGTCGCCG
SEQ ID NO: 9-ASO Ex1b-1
CGGCGACCAGCTGATG
SEQ ID NO: 10-Exon 1b target 2
CTTGAAGGGGACACCA
SEQ ID NO: 11-ASO Ex1b-4
TGGTGTCCCCTTCAAG
SEQ ID NO: 12-Exon 1b target 3
GGATGGTGGCTTACGC
SEQ ID NO: 13-ASO Ex1b-17
GCGTAAGCCACCATCC
SEQ ID NO: 14-RPLP1P4
CCTTCCCTCAGCTGCCGCCAAGCTGCTCGGTCCTTCCGAGGAAGCTAAGGCCGCGTT
GGGGTAAGGCCCTCACTTCATCCTGCGACTAGCACCGCGTCCGGCAGCGCCTGCCCT
ACACTCGCCCGTGCCATGGCCTCCGTCTCCAAGCTCGCCTGCATCTACTCGGCCCTC
ATTCTGCACGACGATGAGGTGACAGTCACGGAGGATAAGATCAATGCCCTCATTAA
AGCAGCCGGTGTAAATGTTGAACCTTTTTGGCCTGGCTTGTTTGCAAAGGCCCTGGC
CAACGTCAACATTGGGAGCCTCATCTGCAATGTAGGGGCTGGTGGACCTGCTCCAGC
AGCTGGTGCTGCACCAGCAGGACGTCCTGCCCCCTCCACTGCTGCTGCTCCAGCTGA
GGAGAAGAAAGTGGAAGCAAAGAAAGAAGAATCCAAGGAGTCTGATGATGACA
SEQ ID NO: 15-BRCA1-Exon 1a
AAAACTGCGACTGCGCGGCGTGAGCTCGCTGAGACTTCCTGGACGGGGGACAGGCT
GTGGGGTTTCTCAGATAACTGGGCCCCTGCGCTCAGGAGGCCTTCACCCTCTGCTCT
GGGTAAAG

### REFERENCES

The following references are herein incorporated by reference in their entireties.
Han, Y. J*., Zhang. J., Lee, J., Mason, J. M., Karginova, O., Yoshimatsu, T., Hao, I., Hurley, I., Brunet. L., Prat, A., Kannanganattu, P., Gack, M. U., Olopade, O. I. The BRCA1Pseudogene Negatively Regulates Anti-Tumor Responses through Inhibition of Innate Immune Defense Mechanisms. Cancer Research, 81(6):1540-1551, 2021.
Han, Y. J., Gack, M. U., Olopade, O. I. Emerging Roles of Pseudogene RNAs in Antitumor and Antiviral Immunity. Journal of Cancer Immunology, 3(2): 104-110, 2021.
Yan B, Wang H, Rabbani Z N, Zhao Y, Li W, Yuan Y, et al. Tumor necrosis factoralpha is a potent endogenous mutagen that promotes cellular transformation. Cancer Res 2006;66:11565-70.
Bhatt D, Ghosh S. Regulation of the NF-kappa B-mediated transcription of inflammatory genes. Front Immunol 2014;5:71.
Lone I N, Shukla M S, Charles Richard J L, Peshev Z Y, Dimitrov S, Angelov D. Binding of NF-kappaB to nucleosomes: effect of translational positioning, nucleosome remodeling and linker histone H1. PLoS Genet 2013; 9: e1003830.
Ashburner B P, Westerheide S D, Baldwin A S Jr. The p65 (RelA) subunit of NF-kappa B interacts with the histone deacetylase (HDAC) corepressors HDAC1 and HDAC2 to negatively regulate gene expression. Mol Cell Biol 2001;21:7065-77.
Lin H, Jiang M, Liu L, Yang Z, Ma Z, Liu S, et al. The long noncoding RNA Lnczc3h7a promotes a TRIM25-mediated RIG-I antiviral innate immune response. Nat Immunol 2019;20:812-23.
Jiang M, Zhang S, Yang Z, Lin H, Zhu J, Liu L, et al. Self-recognition of an inducible host lncRNA by RIG-I feedback restricts innate immune response. Cell 2018;173:906-19.
Demaria O. Cornen S, Daeron M, Morel Y, Medzhitov R, Vivier E. Harnessing innate immunity in cancer therapy. Nature 2019;574:45-56.
Brody J D, Ai W Z, Czerwinski D K. Torchia J A, Levy M, Advani R H, et al. In situ vaccination with a TLR9 agonist induces systemic lymphoma regression: a phase I/II study. J Clin Oncol 2010;28:4324-32.
Demaria O. De Gassart A, Coso S, Gestermann N, Di Domizio J, Flatz L, et al. STING activation of tumor endothelial cells initiates spontaneous and therapeutic antitumor immunity. Proc Natl Acad Sci U S A 2015;112: 15408-13.
Dowling D J, Tan Z, Prokopowicz Z M, Palmer C D, Matthews M A, Dietsch G N, et al. The ultra-potent and selective TLR8 agonist VTX-294 activates human newborn and adult leukocytes. PLoS ONE 2013;8:e58164.
Caskey M, Lefebvre F, Filali-Mouhim A, Cameron M J, Goulet J P, Haddad E K, et al. Synthetic double-stranded RNA induces innate immune responses similar to a live viral vaccine in humans. J Exp Med 2011;208:2357-66.
Gujar S, Pol J G, Kim Y. Lee P W, Kroemer G. Antitumor benefits of antiviral immunity: an underappreciated aspect of oncolytic virotherapies. Trends Immunol 2018;39:209-21.
Puget N, Gad S, Perrin-Vidoz L, Sinilnikova O M, Stoppa-Lyonnet D, Lenoir G M, et al. Distinct BRCA1 rearrangements involving the BRCA1 pseudogene suggest the existence of a recombination hot spot. Am J Hum Genet 2002;70:858-65.
Khanna K K, Jackson S P. DNA double-strand breaks: signaling, repair and the cancer connection. Nat Genet 2001;27:247-54.
Scully R, Ganesan S, Vlasakova K. Chen J, Socolovsky M, Livingston D M. Genetic analysis of BRCA1 function in a defined tumor cell line. Mol Cell 1999;4:1093-9.
Miki Y. Swensen J, Shattuck-Eidens D, Futreal P A, Harshman K. Tavtigian S, et al. A strong candidate for the breast and ovarian cancer susceptibility gene BRCA1. Science 1994;266:66-71.
Han Y J, Boatman S M, Zhang J, Du X C. Yeh A C, Zheng Y, et al. LncRNA BLAT1 is upregulated in basal-like breast cancer through epigenetic modifications. Scientific Reports 2018;8:15572.
Ran F A, Hsu P D, Wright J, Agarwala V, Scott D A, Zhang F. Genome engineering using the CRISPR-Cas9 system. Nat Protoc 2013;8:2281-308.
Parker J S. Mullins M, Cheang M C, Leung S. Voduc D. Vickery T, et al. Supervised risk predictor of breast cancer based on intrinsic subtypes. J Clin Oncol 2009;27:1160-7.
Kato H, Sato S, Yoneyama M. Yamamoto M, Uematsu S, Matsui K, et al. Cell type-specific involvement of RIG-I in antiviral response. Immunity 2005;23: 19-28.
Reikine S, Nguyen J B, Modis Y. Pattern recognition and signaling mechanisms of RIG-I and MDA5. Front Immunol 2014;5:342.
Czerkies M. Korwek Z, Prus W, Kochanczyk M, Jaruszewicz-Blonska J, Tudelska K, et al. Cell fate in antiviral response arises in the crosstalk of IRF, NF-kappa B and JAK/STAT pathways. Nat Commun 2018;9:493.
Wajant H, Pfizenmaier K, Scheurich P. Tumor necrosis factor signaling. Cell Death Differ 2003;10:45-65.
Voltan R, Secchiero P, Casciano F, Milani D, Zauli G, Tisato V. Redox signaling and oxidative stress: cross talk with TNF-related apoptosis inducing ligand activity. Int J Biochem Cell Biol 2016;81:364-74.
Kamata H, Honda S. Maeda S, Chang L, Hirata H, Karin M. Reactive oxygen species promote TNF alpha-induced death and sustained JNK activation by inhibiting MAP kinase phosphatases. Cell 2005;120:649-61.
Wheelhouse N M, Chan Y S, Gillies S E, Caldwell H, Ross J A, Harrison D J, et al. TNF-alpha induced DNA damage in primary murine hepatocytes. Int J MolMed 2003;12:889-94.
Chiang J J, Sparrer K M J, van Gent M, Lassig C, Huang T, Osterrieder N, et al. Viral unmasking of cellular 5S rRNA pseudogene transcripts induces RIG-I-mediated immunity. Nat Immunol 2018;19:53-62.
Rapicavoli N A, Qu K, Zhang J, Mikhail M, Laberge R M, Chang H Y. A mammalian pseudogene lncRNA at the interface of inflammation and anti-inflammatory therapeutics. Elife 2013;2:e00762.
Barker D F, Liu X, Almeida E R. The BRCA1 and 1A1.3B promoters are parallel elements of a genomic duplication at 17q21. Genomics 1996;38:215-22.
Brown M A, Xu C F, Nicolai H, Griffiths B. Chambers J A, Black D, et al. The 50 end of the BRCA1 gene lies within a duplicated region of human chromosome 17q21. Oncogene 1996;12:2507-13.
Pettigrew C A, French J D, Saunus J M, Edwards S L, Sauer A V, Smart C E, et al. Identification and functional analysis of novel BRCA1 transcripts, including mouse Brcal-Iris and human pseudo-BRCA1. Breast Cancer Res Treat 2010;119:239-47.
Minn A J. Interferons and the immunogenic effects of cancer therapy. Trends Immunol 2015;36:725-37.
Wies E, Wang M K, Maharaj N P, Chen K, Zhou S, Finberg R W, et al. Dephosphorylation of the RNA sensors RIG-I and MDA5 by the phosphatase PP1 is essential for innate immune signaling. Immunity 2013;38:437-49.
Rehwinkel J, Gack M U. RIG-I-like receptors: their regulation and roles in RNA sensing. Nat Rev Immunol 2020;20:537-51.
Schoggins J W, Rice C M. Interferon-stimulated genes and their antiviral effector functions. Curr Opin Virol 2011;1:519-25.
Sistigu A, Yamazaki T, Vacchelli E, Chaba K, Enot D P, Adam J, et al. Cancer cell autonomous contribution of type I interferon signaling to the efficacy of chemotherapy. Nat Med 2014;20:1301-9.
Balakirev E S, Ayala F J. Pseudogenes: are they “junk” or functional DNA? Annu Rev Genet 2003:37:123-51.
Zhang Z, Harrison P M, Liu Y, Gerstein M. Millions of years of evolution preserved: a comprehensive catalog of the processed pseudogenes in the human genome. Genome Res 2003:13:2541-58.

1. A method of treating or preventing a non-breast cancer in a subject comprising administering an inhibitor of BRCA1P1 pseudogene expression or activity to a subject in need thereof.

2. The method of claim 1, wherein the subject suffers from a non-breast cancer.

3. The method of claim 1, wherein the subject is at elevated risk of a non-breast cancer based on one or more risk factors.

4. The method of claim 3, wherein the risk factor is selected from: a family history of cancer, being in remission form cancer, environmental or behavioral risk factors, or having a mutation or susceptibility factor that places the subject at increased cancer risk.

5. The method of claim 1, wherein the inhibitor of BRCA1P1 pseudogene expression or activity comprises a nucleic acid that inhibits expression of the BRCA1P1 pseudogene.

6. The method of claim 5, wherein the nucleic acid that inhibits expression of the BRCA1P1 pseudogene is capable of hybridizing with a target sequence within the BRCA1P1 pseudogene.

7. The method of claim 6, wherein the nucleic acid that inhibits expression of the BRCA1P1 pseudogene is capable of hybridizing to a portion of SEQ ID NO: 1 under physiological conditions.

8. The method of claim 7, wherein the nucleic acid that inhibits expression of the BRCA1P1 pseudogene is capable of hybridizing to a portion of SEQ ID NOS: 2 or 3 under physiological conditions.

9. The method of claim 8, wherein the nucleic acid that inhibits expression of the BRCA1P1 pscudogene is capable of hybridizing to all or a portion of one of SEQ ID NOS: 4, 6, 8, 10, and 12, under physiological conditions.

10. The method of claim 5, wherein the nucleic acid that inhibits expression of the BRCA1P1 pseudogene is an antisense oligonucleotide (ASO), an siRNA, and shRNA, or an element of a Cas/CRISPR system.

11. The method of claim 10, wherein the nucleic acid that inhibits expression of the BRCA1P1 pseudogene is an ASO.

12. The method of claim 11, wherein the ASO is capable of hybridizing with a target sequence within the BRCA1P1 pseudogene.

13. The method of claim 12, wherein the ASO is capable of hybridizing to a portion of SEQ ID NO: 1 under physiological conditions.

14. The method of claim 13, wherein the ASO is capable of hybridizing to a portion of SEQ ID NOS: 2 or 3 under physiological conditions.

15. The method of claim 14, wherein ASO is capable of hybridizing to all or a portion of one of SEQ ID NOS: 4, 6, 8, 10, and 12, under physiological conditions.

16. The method of claim 12, wherein the ASO comprises 70% sequence identity to one of SEQ ID NOS: 5, 7, 9, 11, and 13.

17. The method of claim 16, wherein the ASO comprises 100% sequence identity to one of SEQ ID NOS: 5, 7, 9, 11, and 13.

18. The method of claim 16, wherein the ASO comprises one or more chemical modifications.

19. The method of claim 18, wherein the ASO comprises a locked nucleic acid (LNA) modification and/or 2′-methoxyethyl (MOE) modification.

20. The method of claim 1, wherein the inhibitor is co-administered with a chemotherapeutic, immunotherapeutic, surgery, and/or radiation.

21. A composition comprising an ASO inhibitor of BRCA1P1.

22. The composition of claim 21, wherein the ASO is capable of hybridizing with a target sequence within the BRCA1P1 pseudogene.

23. The composition of claim 22, wherein the ASO is capable of hybridizing to a portion of SEQ ID NO: 1 under physiological conditions.

24. The composition of claim 23, wherein the ASO is capable of hybridizing to a portion of SEQ ID NOS: 2 or 3 under physiological conditions.

25. The composition of claim 24, wherein ASO is capable of hybridizing to all or a portion of one of SEQ ID NOS: 4, 6, 8, 10, and 12, under physiological conditions.

26. The method of claim 22, wherein the ASO comprises 70% sequence identity to one of SEQ ID NOS: 5, 7, 9, 11, and 13.

27. The composition of claim 26, wherein the ASO comprises 100% sequence identity to one of SEQ ID NOS: 5, 7, 9, 11, and 13.

28. The composition of claim 26, wherein the ASO comprises one or more chemical modifications.

29. The composition of claim 28, wherein the ASO comprises a locked nucleic acid (LNA) modification and/or 2′-methoxyethyl (MOE) modification.
